(19) United States
(12) Patent Application Publication
(10) Pub. No.: US 2013/0237898 A1
(43) Pub. Date: Sep. 12, 2013

(54) WOUND TREATMENT SYSTEM AND METHOD

(71) Applicants: Jeffrey B. Kirkham, Salt Lake City, UT (US); Joshua C. Vandenbrink, Temecula, CA (US)

(72) Inventors: Jeffrey B. Kirkham, Salt Lake City, UT (US); Joshua C. Vandenbrink, Temecula, CA (US)

(21) Appl. No.: 13/413,339

(22) Filed: Mar. 6, 2012

(51) Int. Cl. A61F 13/84

(52) U.S. Cl. 604/15

(57) ABSTRACT

The present invention extends to a wound treatment system and a method of using the wound treatment system to treat a flesh wound such as from a gunshot, knife, or shrapnel. The wound treatment system comprises an applicator that contains absorbent material, and a plunger. The applicator is configured to be inserted into a flesh wound to inject the absorbent material directly at the source of bleeding. In this manner, the absorbent material is less likely to become contaminated and can apply direct internal pressure to the source of bleeding to mitigate the risk of serious injury or death from blood loss.

### CROSS-REFERENCE TO RELATED APPLICATIONS

Not Applicable.

### BACKGROUND

### Background and Relevant Art

When a person suffers a traumatic wound, such as a wound from a gunshot, knife, or shrapnel, the primary source of bleeding, (i.e. vein/artery) is often located deep inside the wound. To stop the bleeding, it is necessary to apply pressure to the wound, at the source of the bleeding. The deeper the wound, the more difficult it can be to apply pressure directly to the source of the bleeding, to effectively stop the bleeding and to treat the wound. For example, when a gunshot wound punctures an artery, it can be difficult to apply pressure directly to the artery, deep within the wound, in order to stop the bleeding. For this reason, deep wounds can result in continuous internal bleeding and can even be lethal.

Various products have been developed and used to treat such wounds. For example, gauze can sometimes be used to pack such wounds by manually injecting the gauze into the wound with one or two fingers, bit-by-bit. By forcing gauze into the wound, it is sometimes possible to apply enough internal pressure to the source of the bleeding in order to stop the bleeding. However, there are several problems that exist with the traditional packing of a wound with gauze.

Initially, it is noted that gauze is often supplied in a bulky roll that makes it inconvenient to be carried by a soldier for use during battle. It can also be difficult to unroll and pack the gauze, during a chaotic battle scene, in an aseptic manner. For example, as the gauze is inserted into the wound, the remaining strand of gauze is often unwound and becomes contaminated by dragging the unwound roll of gauze on the ground or across dirty clothing. Additionally, the person who inserts the gauze must do so manually (e.g. by inserting a finger into the wound to push the gauze into the wound). Accordingly, any contaminants on the person's hands will likely be transferred to the gauze and/or the wound as it is being packed into the wound.

Furthermore, it can be very difficult for the person who is inserting the gauze to properly direct the gauze through the wound, to the deepest sources of the bleeding. For example, as the person is packing the gauze into the wound with his finger(s), the gauze can become bunched inside the wound before it reaches the deepest part of the wound. This is particularly problematic when packing shrapnel wounds, punctures and gunshot wounds that present irregular passageways and caverns. It can be difficult to fully and adequately pack a wound that spread across an irregular wound area that is deep and narrow, as opposed to an open laceration or clean puncture or gunshot wound. In many instances, the gauze is only packed into a portion of the wound, resulting in internal bleeding that can cause further injury or sometimes death. Contaminants that are introduced during the packing can also cause additional injury.

The difficulty in maintaining asepsis during the gauze packing procedure is even further exacerbated when the packing is performed by the injured party. This is because the wounded person must use one hand to insert the gauze into the wound and the other hand to hold and unravel the roll or folds of gauze. Notably, it can be difficult to unravel a roll of gauze with one hand, particularly when injured, such that it is even more likely that the gauze will fall onto or come into contact with an unclean material or surface (including a bloody and dirty hand of the injured party).

Finally, it is also noted that packing a wound with gauze can also take an undesired amount of time, resulting in excessive blood loss when attempting to perform the procedure in asepsis.

Although applicator delivery systems that are used with granular haemostatic agents can mitigate some of the risk of contaminating wounds during treatment, they also suffer from various other shortcomings. For example, the haemostatic agent, when coagulated, is structurally insufficient to apply the necessary pressure within the wound to stop the bleeding. While external surface pressure can be applied, after inserting the haemostatic agent, the surface pressure is often insufficient to stop the bleeding at the source, deep within the wound.

Gauze can also be packed into the wound after inserting the granules, to provide substance for applying the necessary internal pressure. However, this reintroduces many of the problems discussed above, during the packing. Furthermore, it has been discovered that granular haemostatic agents, such as Celox, can enter the blood stream and form blood clots in the body, away from the wound, such as is the brain, heart, or other organs.

Additionally anything introduced into a wound will have to be removed prior to surgical closure of the wound at the hospital. Granule products have been noted to be very difficult to debride wounds and tend to adhere to tissue inside the wound itself.

### BRIEF SUMMARY

The present invention extends to a wound treatment system and a method of using the wound treatment system to treat a wound. The wound treatment system comprises an applicator that contains an absorbent material, and a plunger. The applicator is formed with an elongate sleeve extending between a plunger end and a deployment end. The absorbent material which is packed, stacked, compressed or otherwise disposed within the elongate sleeve has a mass that is continuously and integrally connected, so as to facilitate its removal from the wound. In some embodiments, the absorbent material has a length (when in an uncompressed state) that is at least three times greater than the length of the sleeve, to facilitate the packing of a wound.

The plunger is used to eject the absorbent material from the deployment end of the applicator. In this manner, the absorbent material can be directly injected into a flesh wound, such as a wound from a gunshot, knife, or shrapnel, in an aseptic manner. Further, because the applicator is inserted into the flesh wound prior to injecting the absorbent material, the wound treatment system facilitates the speedy injection of an absorbent material directly at the source of bleeding, even deep within the wound, thus facilitating the application of pressure and foreign bodies to promote clotting in order to stop blood loss.

Preferably, the applicator has a tip or deployment end that is tapered to facilitate the injection of the applicator into the wound. The applicator can also be formed from flexible materials that facilitate the navigation of the applicator into irregular wounds.

Different absorbent materials can also be used, with different decompression properties and forms, so as to facilitate their use in packing a wound, as described herein. The absorbent materials can also be impregnated or otherwise treated with chemical agents, such as haemostatic agents to assist the body in clotting the blood, or anti-microbial agents to prevent infection. In some embodiments, mechanical materials are also used, such as x-ray detectable materials, within the absorbent materials, so as to facilitate an exam to determine whether the entire absorbent material has been removed prior to cleaning and closing the wound at a later time.

The applicator can be covered with a cap (such as at the deployment end) to help keep the absorbent material within the sleeve and to protect the absorbent material from contaminants during periods of non-use. The entire applicator can also be packaged within a protective sleeve for similar purposes.

Methods for using the inventive treatment system include deploying the absorbent material from one or more applicators into the wound. In some instances, the wound is further packed with gauze or another packing material, on top of the absorbent material. In other methods, the absorbent material is deployed onto a surface wound and then wrapped with gauze or another wrapping material or compression bandage.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates a prior art wound treatment system for injecting a granular agent into a puncture wound;

FIG. 2 illustrates an applicator of a wound treatment system that can be used in embodiments of the invention;

FIG. 3 illustrates some absorbent materials of various form factors that can be included within the wound treatment system of the present invention;

FIGS. 4A-4B illustrate some exemplary configurations of the wound treatment system of the present invention;

FIG. 5 illustrates an exemplary wound treatment system after absorbent material having a form of a string or cord has been ejected or deployed from the applicator;

FIGS. 6A-6D illustrate various stages of a deep tissue wound being treated by a wound treatment system of the invention; and

FIGS. 7A-7C illustrate exemplary retaining means for retaining the absorbent material within the applicator of the wound treatment system.

### DETAILED DESCRIPTION

The present invention extends to a wound treatment system and a method of using the wound treatment system to treat a wound. The wound treatment system comprises an applicator that contains an absorbent material, and a plunger. The applicator is formed with an elongate sleeve extending between a plunger end and a deployment end. The absorbent material which is packed, stacked, compressed or otherwise disposed within the elongate sleeve has a mass that is continuously and integrally connected, so as to facilitate its removal from the wound. In some embodiments, the absorbent material has a length (when in an uncompressed state) that is at least three times greater than the length of the sleeve, to facilitate the packing of a wound.

The plunger is used to eject the absorbent material from the deployment end of the applicator. In this manner, the absorbent material can be directly injected into a flesh wound, such as a wound from a gunshot, knife, or shrapnel, in an aseptic manner. Further, because the applicator is inserted into the flesh wound prior to injecting the absorbent material, the wound treatment system facilitates the speedy injection of an absorbent material directly at the source of bleeding, even deep within the wound, thus facilitating the application of pressure and foreign material to stop blood loss.

Preferably, the applicator has a tip or deployment end that is tapered to facilitate the injection of the applicator into the wound. The applicator can also be formed from flexible materials that facilitate the navigation of the applicator into irregular wounds.

Different absorbent materials can also be used, with different decompression properties and forms, so as to facilitate their use in packing a wound, as described herein. The absorbent materials can also be impregnated or otherwise treated with chemical agents, such as hemostatic agents to assist the body in clotting the blood or anti-microbial agents to prevent infection. In some embodiments, mechanical materials are also used, such as x-ray detectable materials, within the absorbent materials, so as to facilitate an exam to determine whether the entire absorbent material has been removed prior to cleaning and closing the wound at a later time.

The applicator can be covered with a cap (such as at the deployment end) to help keep the absorbent material within the sleeve and to protect the absorbent material from contaminants during periods of non-use. The entire applicator can also be packaged within a protective sleeve for similar purposes.

Methods for using the inventive treatment system include deploying the absorbent material from one or more applicators into the wound. In some instances, the wound is further packed with gauze or another packing material, on top of the absorbent material. In other methods, the absorbent material is deployed onto a surface wound and then wrapped with gauze or another wrapping material.

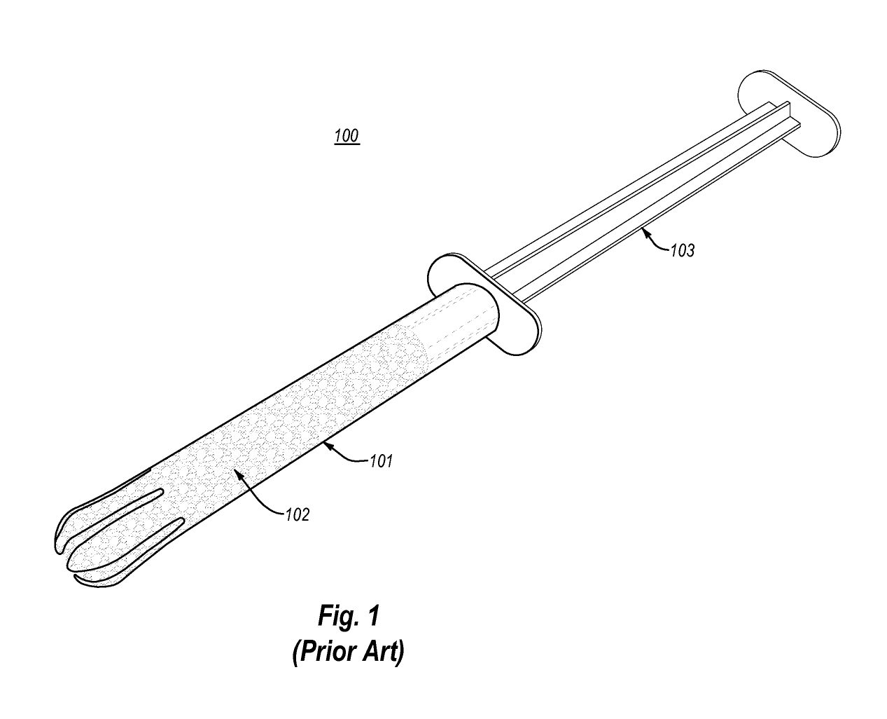

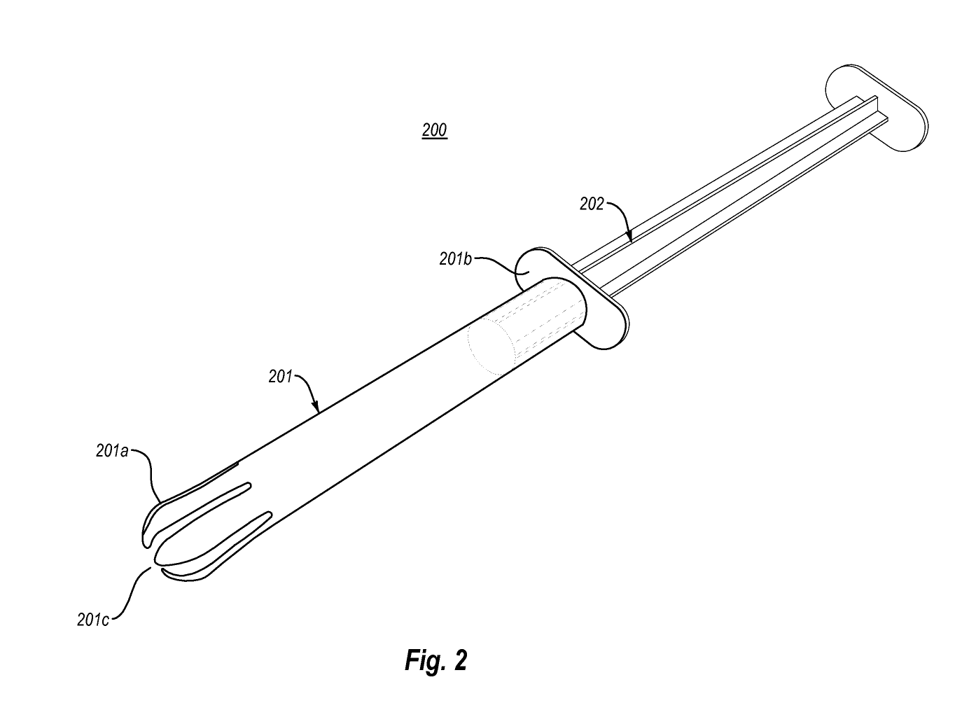

FIG. 2 illustrates some aspects of an exemplary wound treatment system 200 according to certain embodiments of the invention. Wound treatment system 200, for example, comprises an applicator 201 and a plunger 202. Applicator 201 and plunger 202 can be similar to those used in wound treatment system 100 shown in FIG. 1. In other words, wound treatment system 200 can be comprised of commonly used applicators and plungers. However, as detailed below, applicator 201 can be designed in various novel ways to further facilitate the injection of absorbent material into a flesh wound.

Applicator 201 is shaped as an elongate sleeve that has a body that is round (as shown), oval, rectilinear, or another shape that is not currently shown. Applicator 201 can also be configured in various lengths or diameters/widths and shapes to accommodate and treat wounds of different depths, widths and shapes.

In some embodiments, the applicator 201 has a length of between about 2 inches and 10 inches, and more preferably a length in a range of between about 4 inches and 8 inches, and even more preferably a length of about 6 inches or in a range of between about 5 inches and 7 inches.

The diameter of the applicator is in a range of between about ¼ inch and 2 inches, and more preferably between about ½ inch and 1 inch.

Applicator 201 can be made of various materials including rigid and flexible materials. For example, applicator 201 can be made of a relatively flexible material to facilitate insertion of applicator 201 into irregularly shaped wounds.

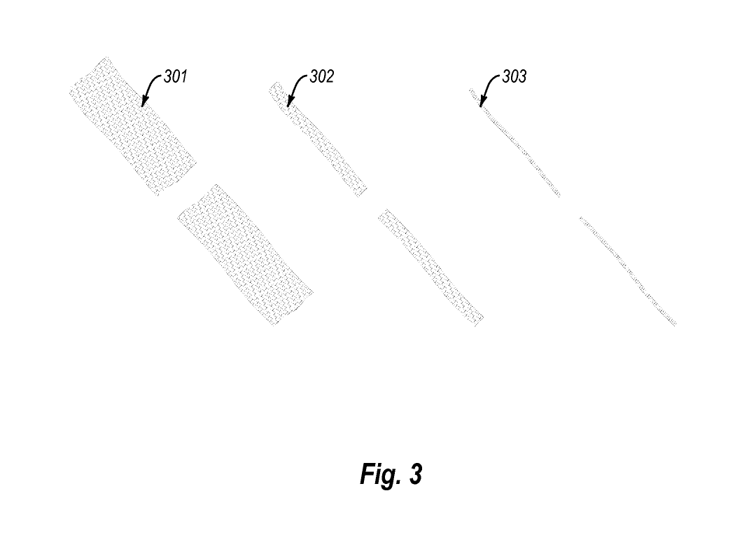

Applicator 201 includes a deployment end 201a, a plunger end 201b, and an opening 201c. Plunger 202 is inserted into the elongate sleeve at plunger end 201b to eject absorbent material contained within the elongate sleeve from the elongate sleeve through opening 201c (such as shown and described in more detail below, in reference to FIGS. 3-5).

Wound treatment system 200 can be packaged with plunger 202 pre-inserted into applicator 201. Alternatively, wound treatment system 200 can be packaged with plunger 202 removed from applicator 201, in which case a cap or other means of sealing plunger end 201b of applicator 201 may be included to retain the absorbent material within the sleeve and to ensure that the absorbent material remains aseptic.

FIG. 3 illustrates exemplary form factors for absorbent materials that can be contained within applicator 201. Absorbent material, as described herein, comprises any hydrophilic material that is suitable for treating wounds. A common example of such a material is gauze or another woven material; however, the invention is not limited to using gauze as the absorbent material.

According to many embodiments of the invention, the absorbent material is compressed within applicator 201 to maximize the amount of absorbent material that can be injected into a flesh wound. This configuration facilitates packaging and portability of gauze for soldiers.

FIG. 3 illustrates that the absorbent material can have different form factors, including different widths and lengths. For example, material 301 represents a woven or sponge like material having a generally large width and/or thickness. Material 301 can comprise gauze, another type of woven material, a sponge or another material.

The thickness of the absorbent material can vary, from less than ¼ inch to more than four inches, for example. Notably, material 301 represents absorbent material that has a width greater than the diameter or width of applicator 201 and, in some instances more than twice or three times the width of the diameter of the applicator.

Material 302 represents a material having a relatively smaller width. For example, material 302 can also comprise gauze having a common width of ¼ or ½ inches. In some instances, material 302 has a width that is substantially the same as the diameter of the applicator.

Material 303 represents a material having a minimal width such as a width of a string or cord, having a generally cylindrical cross-sectional. For example, material 303 can comprise a string, cord, rope or other segment of a woven material (like gauze) or another fibrous and continuously connected or interwoven material. In some embodiments, material 303 has a width or diameter that is less than the diameter of the applicator and, in some instances, less than ½ the diameter of the applicator.

Although the above description provides various specific exemplary widths, it will be appreciated that absorbent material of any reasonable width may be used.

In some embodiments, the absorbent material contained within applicator 201 also has a length that is at least equal to the length of the applicator and, in most embodiments, greater than the length of the applicator. One reason for this is to provide enough material to be applied inside of a wound to apply pressure at the point of bleeding and to assist in stopping the bleeding.

In some embodiments, the length of the absorbent material is at least three times greater than the length of applicator 201. The length of the absorbent material can also vary to accommodate different needs and preferences, such as, for example, based on the width and type of the absorbent material. For example, for a wider absorbent material, a piece of absorbent material having a length of approximately 3 feet or less may be used. In contrast, for absorbent material comprising a string, the length of the absorbent material may exceed 20, 50 or even 100 feet in length. Generally speaking, the absorbent material will have a length of between about 1 foot and 100 feet.

Generally, a single continuous strand of absorbent material is contained within applicator 201 to facilitate the removal of the absorbent material from the flesh wound after treatment. However, in some embodiments, multiple pieces of absorbent material can also be contained within applicator 201.

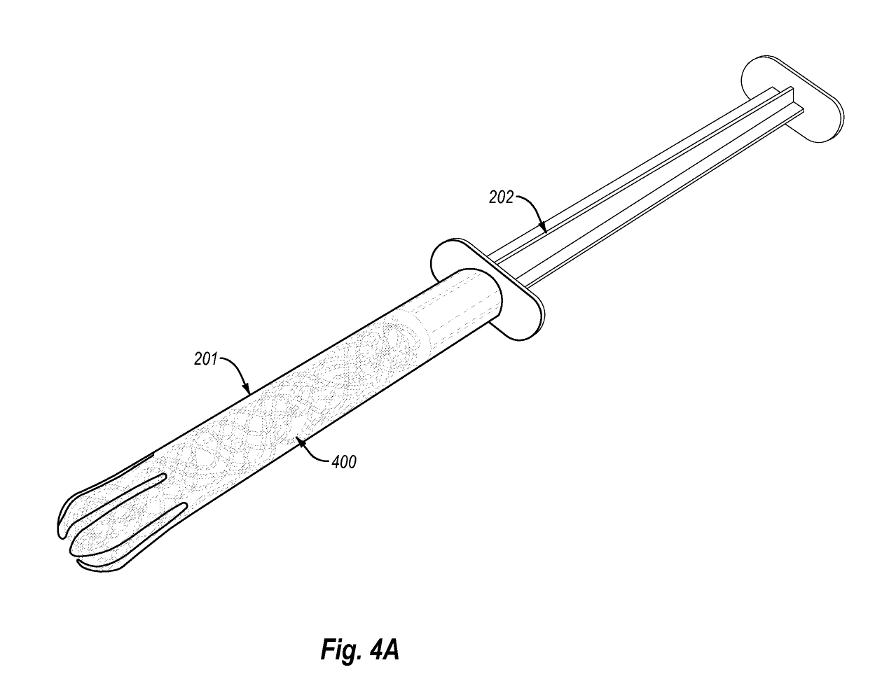

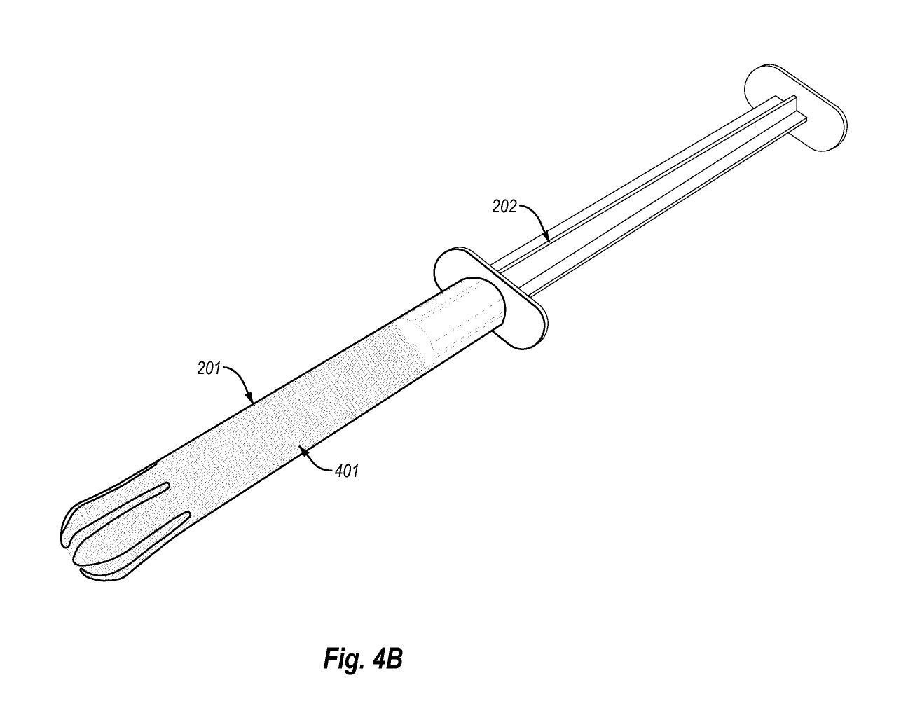

FIG. 4A illustrates a transparent view of applicator 201 to illustrate absorbent material 400 compressed within applicator 201. Absorbent material 400, as shown, can be packed and/or compressed into applicator 201 in a random manner. Alternatively, as shown in FIG. 4B, absorbent material 401 can be packed into applicator 201 using a repeating pattern, such as a folded pattern, to maximize the amount of absorbent material that can be contained/compressed within applicator 201. Using a folded repeating pattern can be most effective when an absorbent material having a width similar to or smaller than the diameter or width of applicator 201 is used.

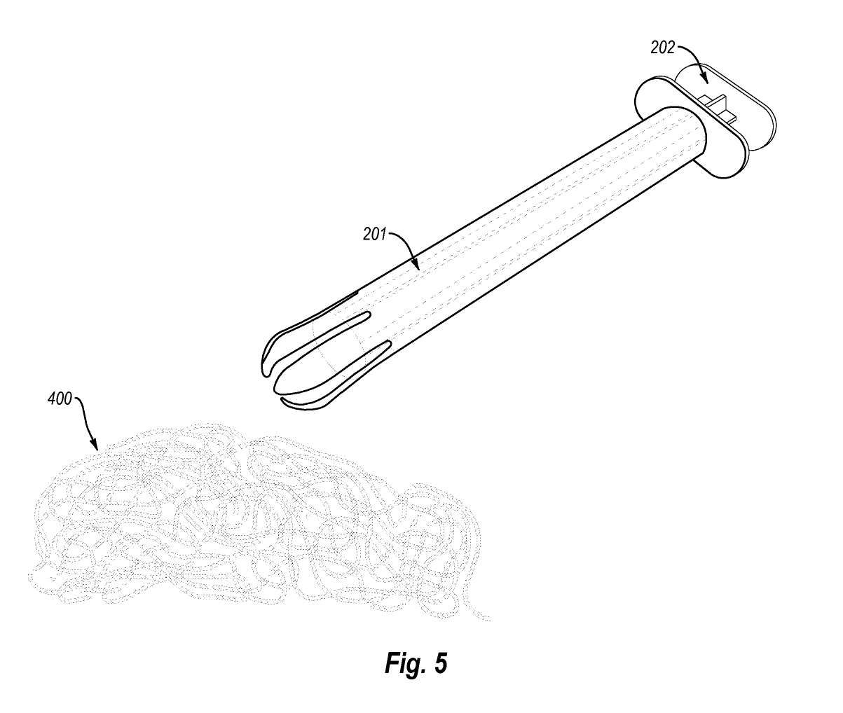

FIG. 5 illustrates absorbent material 400 after it has been ejected from applicator 201 by pressing the plunger 202 into the applicator 201. As shown, once deployed, the absorbent material 400 expands from its compressed state to occupy a greater area than it contained within the applicator. In some cases, the amount of area the absorbent material occupies (in an uncompressed state), is at least 25%, 50%, or more than 100% greater than the area it occupied within the applicator.

Because absorbent material 400 can be ejected after applicator 201 is inserted into a flesh wound, absorbent material 400 can be positioned directly at the source of the bleeding, even at the base of deep wounds. Also, as opposed to the insertion of gauze by hand, applicator 201 facilitates the aseptic deployment and spreading of the absorbent material 400 throughout the area of the wound to ensure the entire source of bleeding is treated. Specifically, because the absorbent material is injected internally, it more easily spreads throughout the wound to apply internal pressure to the wound.

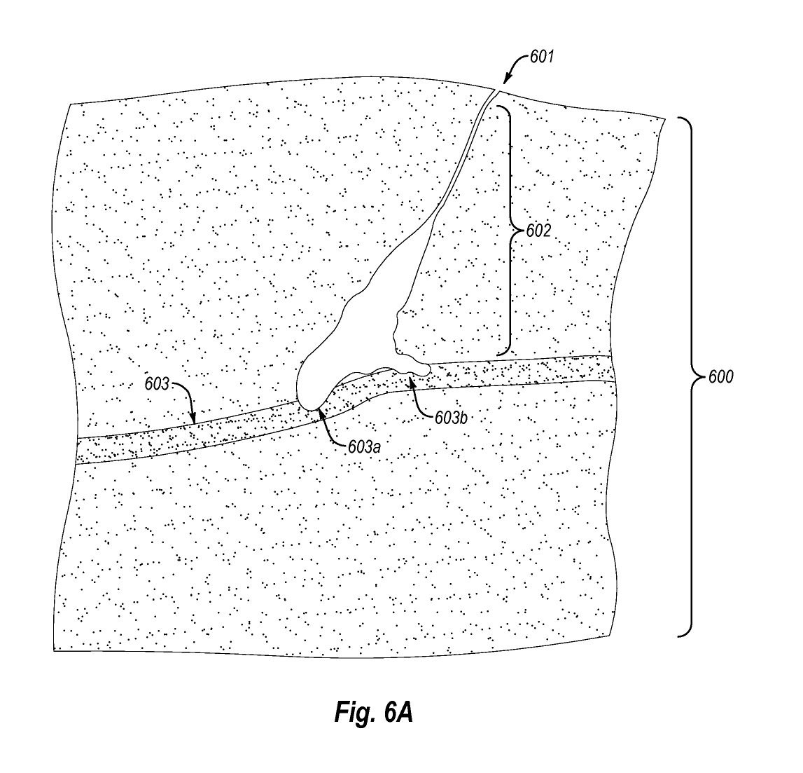

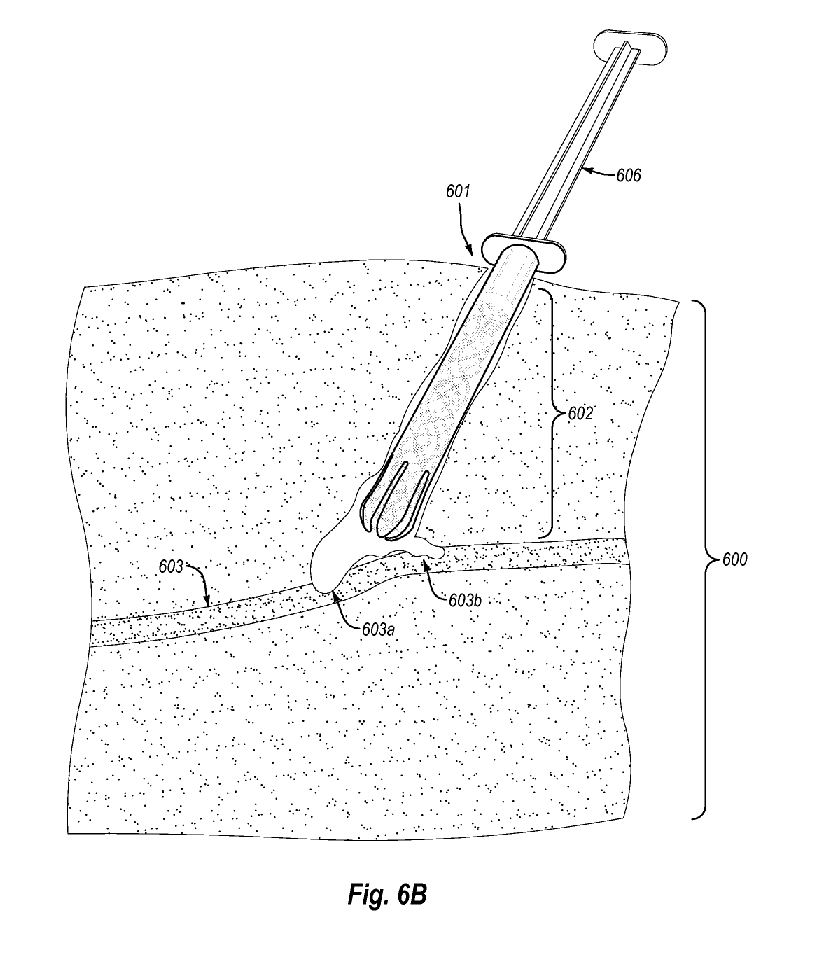

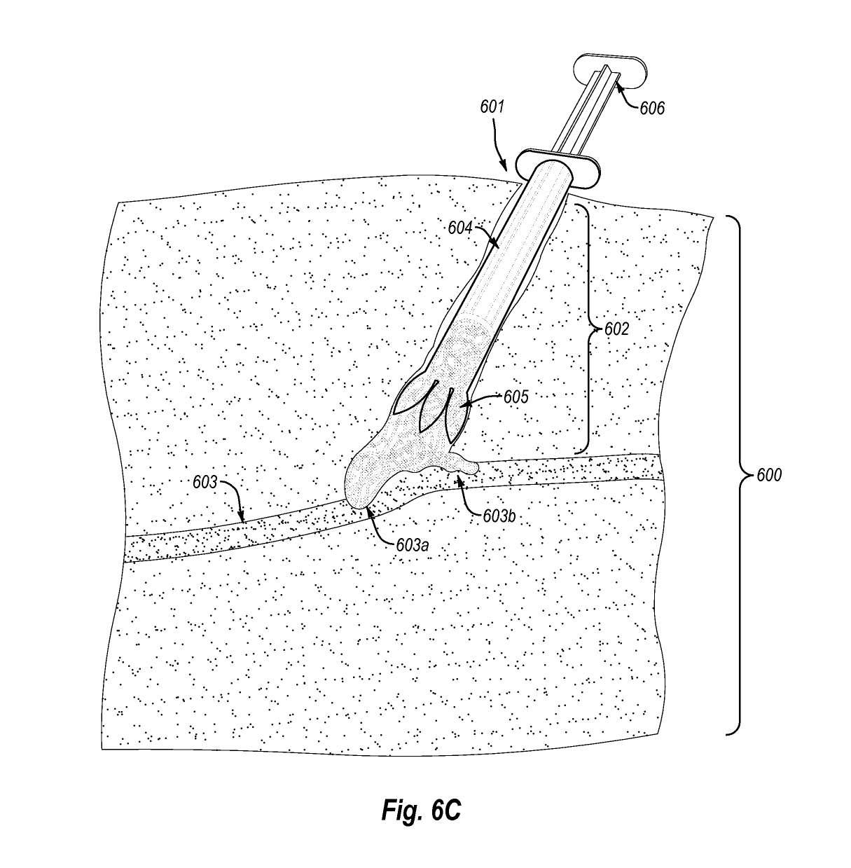

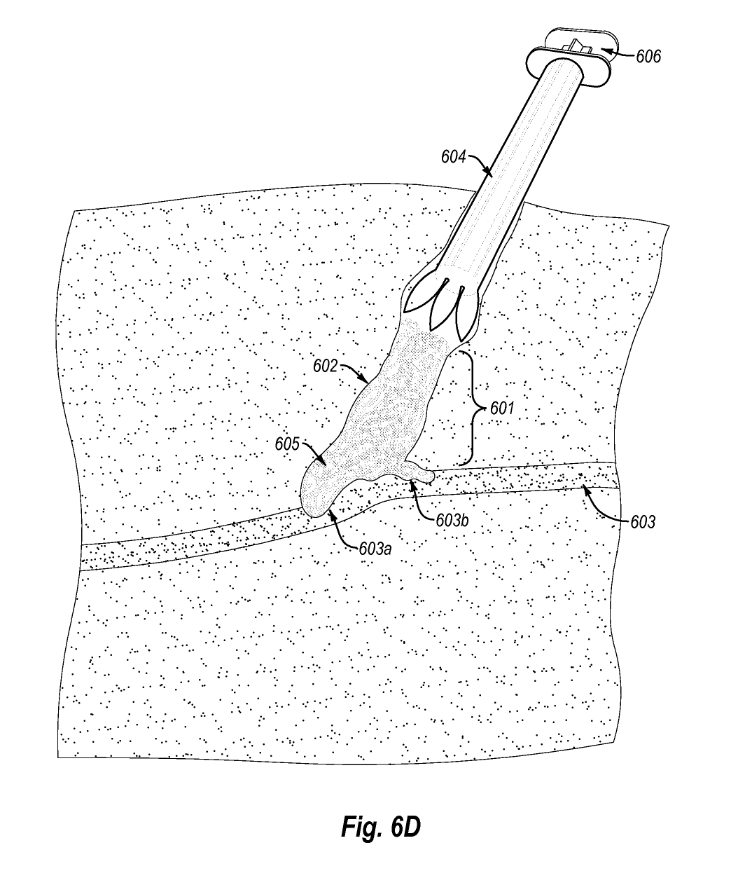

FIG. 6A illustrates an exemplary flesh wound 601, in the leg 600 of a victim, which may be treated using the wound treatment system of the present invention. Wound 601 includes a puncture 602 into leg 600. Puncture 602 can be from a gunshot, a knife, shrapnel, or any other source. As shown, puncture 602 can extend deep into leg 600 and can have an irregular shape, thus making it difficult or impossible to reach the deepest source of bleeding with one's fingers. A wound or puncture from a gunshot, knife, shrapnel, and the like can pierce an artery or vessel in different locations. For example, as shown in FIG. 6A, wound 601 has pierced artery 603 in two locations, 603a and 603b. It can be difficult to reach multiple locations when inserting gauze using one's fingers.

Accordingly, to facilitate treatment of wound 601, the applicator 604 of a treatment system can be inserted into wound 601 via puncture 602 as shown in FIG. 6B. Applicator 604 can be the same as applicator 201. Applicator 604 contains absorbent material 605. Although absorbent material 605 is shown as being similar to material 303 of FIG. 3, any suitable absorbent material can be used. Applicator 604 can be inserted into puncture 602 until applicator 604 is in the immediate proximity of the source of bleeding (e.g. pierces 603a and 603b), deep within the wound 601.

To facilitate different wound depths, different applicators of different lengths can also be utilized, as described above. During treatment, for example, a practitioner can be presented with a kit of different treatment systems/applicators having different shapes and/or sizes and that contain similar or different types of absorbent materials.

FIG. 6C illustrates that absorbent material 605 has been partially ejected from applicator 604 by depressing plunger 606. Notably, absorbent material 605 has spread into each pierce 603a and 603b as well as throughout wound 601. Also, as the remaining portion of absorbent material 605 is ejected from applicator 604, additional pressure will be applied to wound 601 to stop blood loss.

FIG. 6D illustrates that absorbent material 605 has now been fully ejected into the wound 601 and that applicator 604 has been partially removed from wound 601. If necessary, an additional one or more wound treatment systems (of the same or different configuration) can be used to treat wound 601. For example, if wound 601 were large thus requiring additional absorbent material, another applicator could be inserted into wound 601 in a similar manner to inject additional absorbent material. Further, after injecting absorbent material using the wound treatment system of the present invention, additional absorbent material can be used to treat wound 601 using traditional techniques, such as by inserting gauze using a finger to increase internal pressure on wound 601, at the upper end of the wound, or by wrapping gauze around leg 600 to apply external surface pressure.

Although the above description describes the use of the wound treatment system to treat a penetrating or puncture wound, the wound treatment system can also be used to treat non-penetrating wounds such as lacerations or avulsions, by deploying the absorbent material directly to the surface area of the wound or into a hand for manual application to the wound. In either event, the wound treatment system provides the benefit of maintaining the absorbent material in asepsis before and often during treatment. The wound treatment systems of the invention also facilitate quick deployment of the absorbent material with accurate placement.

Another advantage of the invention in treating wounds of any type is that the wound treatment system facilitates self-treatment. For example, it is generally easier for a person to inject absorbent material into himself rather than packing gauze by hand. Also, the wound treatment system better maintains the absorbent material in asepsis because the absorbent material is not easily exposed to contaminants until it is ejected from the applicator.

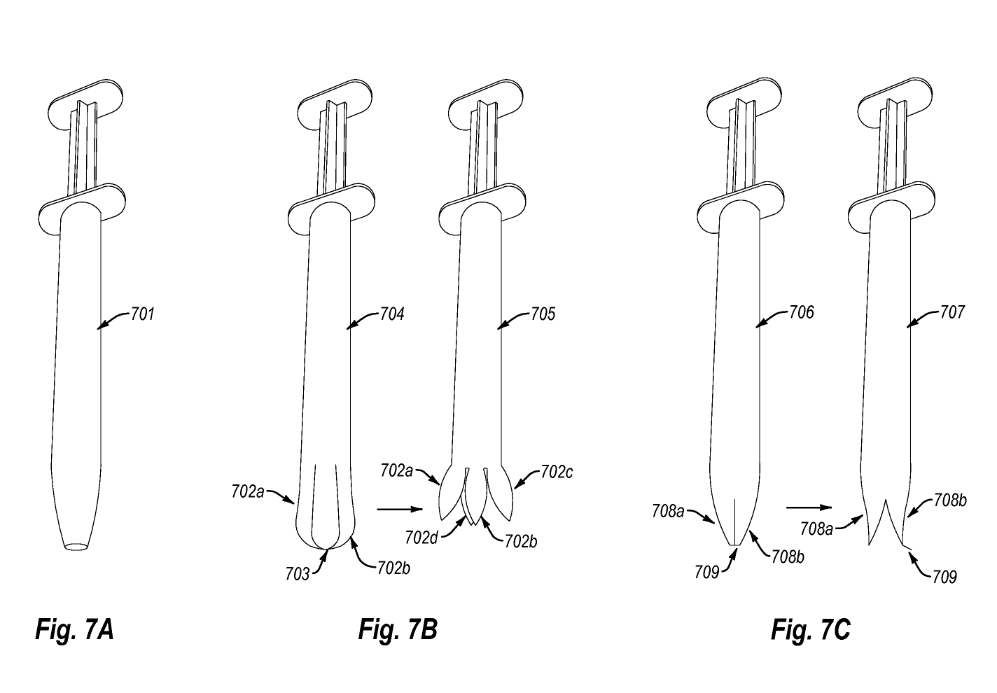

FIGS. 7A-7C illustrate various designs for various retaining means that can be used for retaining the absorbent material within the applicator of a wound treatment system of the invention. Retaining means comprise means for retaining compressed absorbent material within the applicator until it is intentionally deployed from the applicator, such as, for example, during treatment (as described above). FIG. 7A illustrates that the retaining means can comprise a tapered deployment end 701. The taper can be sufficient to retain compressed absorbent material within the applicator until a predetermined pressure is applied to the plunger. The taper can also facilitate placing the applicator into and navigating the applicator through a wound.

FIG. 7B illustrates that the retaining means can also comprise a number of flaps at the deployment end. FIG. 7B includes a closed configuration 704 and an open configuration 705. Flaps 702a and 702b are shown in closed configuration 704 as being interconnected via a seam 703. Seam 703 is designed to rupture when a predetermined pressure is applied to the plunger as shown in open configuration 705. Specifically, in open configuration 705 flaps 702a-702d are shown as separated from each other to increase the size of the opening. In this manner, injection of the absorbent material is facilitated. Although FIG. 7B shows the retaining means as comprising four flaps, the retaining means can comprise other numbers of interconnected flaps (e.g. two, three, or more). The flaps can also be sealed shut with an adhesive, molding, welding or other manufacturing process.

FIG. 7C illustrates that the retaining means can comprise a bridge for securing a number of flaps. FIG. 7C includes a closed configuration 706 and an open configuration 707. In closed configuration 706, a first flap 708a is connected to a second flap 708b via bridge 709. Bridge 709 can be made of the same or different material as the remainder of the applicator. Bridge 709 is configured to rupture when a predetermined pressure is applied to the plunger. For example, as shown in open configuration 707, bridge 709 has ruptured from first flap 708a thus enabling first flap 708a and second flap 708b to separate. Bridge 709 is shown as being permanently connected to second flap 708b so that bridge 709 does not remain within a wound after injection of absorbent material. The bridge 709 can also substantially cover (in a sealed or unsealed configuration) the entire opening of the applicator, so as to further protect the aseptic condition of the absorbent material.

A seam can also be provided between first flap 708a and second flap 708b to create a seal. Such a seam could be configured similar to seam 703 of FIG. 7B to rupture when the predetermined pressure is applied. However, because of bridge 709, a seam is not necessary to retain the compressed absorbent material within the applicator.

Although FIG. 7C shows two flaps, a bridge can be used to secure more than two flaps. For example, a bridge can be permanently connected to a first flap while being temporarily connected to two or more other flaps. Accordingly, when the predetermined pressure is applied to the plunger, the bridge can rupture from the two or more other flaps while remaining connected to the first flap.

Regardless of how the applicator is configured (i.e. whether retaining means are used or not), a friction fit cap can also be used to cover the deployment end of the applicator. The cap (not shown) can be beneficial to prevent the unintentional deployment of the absorbent material and/or to prevent contamination of the absorbent material.

In some embodiments, the retaining means can comprise the cap. For example, the cap can be pressed over the deployment end of the applicator to retain the absorbent material within the applicator until the applicator is inserted into the wound.

In some embodiments, the absorbent material can be impregnated or coated with a haemostatic agent to assist in the clotting of blood. Additionally, in some embodiments, the absorbent material can be impregnated, coated, or interwoven with a material that is detectable via x-ray, radio waves, or some other means to facilitate detection of the absorbent material to ensure complete removal from the wound.

The wound treatment system can also comprise a sealed envelope or other package containing the applicator and plunger. The applicator and plunger can be packaged separately inside the sealed package or the plunger can be pre-inserted into the plunger end of the applicator. The sealed package can assist in preventing contamination of the absorbent material within the applicator.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A wound treatment system comprising:
an applicator comprising an elongate sleeve having a diameter and extending a length between a plunger end and a deployment end;
an absorbent material disposed within the applicator, the absorbent material having a length that is at least twice the length of the sleeve; and
a plunger for ejecting the absorbent material from the deployment end of the applicator.

2. The wound treatment system of claim 1, wherein the absorbent material is disposed within the sleeve in a compressed state.

3. The wound treatment system of claim 1, wherein the absorbent material is disposed within the sleeve in a folded repeating pattern.

4. The wound treatment system of claim 1, wherein the absorbent material has a length between 3 and 100 feet.

5. The wound treatment system of claim 1, wherein the absorbent material is a woven material having a length of at least 5 feet.

6. The wound treatment system of claim 1, wherein the absorbent material has a width that is greater than the diameter of the sleeve.

7. The wound treatment system of claim 1, wherein the absorbent material is a single continuous length of gauze.

8. The wound treatment system of claim 1, wherein the applicator further comprises a retaining means for retaining the absorbent material within the sleeve until a predetermined pressure is applied to the plunger positioned within the sleeve.

9. The wound treatment system of claim 8, wherein the retaining means comprises a tapered portion of the deployment end.

10. The wound treatment system of claim 9, wherein the tapered portion comprises a number of flaps that are at least partially interconnected in a manner that allows each flap to separate upon the predetermined pressure being applied to the plunger thus creating a larger opening at the deployment end through which the absorbent material exits the sleeve.

11. The wound treatment system of claim 10, wherein each flap is connected to the adjacent flaps.

12. The wound treatment system of claim 10, wherein the number of flaps are at least partially interconnected via a bridge.

13. The wound treatment system of claim 1, wherein the applicator further comprises a cap that is detachably connected to the deployment end and covers the deployment end.

14. The wound treatment system of claim 1, further comprising a protective envelope for containing the applicator and the plunger in an aseptic manner.

15. The wound treatment system of claim 1, wherein the absorbent material is coated or impregnated with a haemostatic agent.

16. The wound treatment system of claim 1, wherein the absorbent material is coated, impregnated or interwoven with a material that is detectable via x-ray or radio waves.

17. The wound treatment system of claim 1, wherein the sleeve of the applicator has a diameter that is within a range of about 0.25 inches and about 0.75 inches.

18. A method for treating a flesh wound by deploying an absorbent material into the flesh wound with a wound treatment system, the method comprising:
accessing a wound treatment system, the wound treatment system comprising:
an applicator comprising an elongate sleeve having a diameter and extending a length between a plunger end and a deployment end;
an absorbent material disposed within the applicator, the absorbent material having either a length that is greater than the length of the sleeve and/or a width that is greater than the diameter of the sleeve; and
a plunger for ejecting the absorbent material from the deployment end of the applicator;
inserting the deployment end of the sleeve into the flesh wound;
pressing the plunger into the plunger end of the sleeve to eject the absorbent material out of the deployment end of the sleeve and into the flesh wound; and
removing the applicator from the flesh wound, wherein the absorbent material remains in the flesh wound.

19. The method of claim 18, wherein the wound treatment system comprises a first wound treatment system, the method further comprising:
accessing a second wound treatment system, the second wound treatment system comprising:
a second applicator comprising a second elongate sleeve having a diameter and extending a length between a second plunger end and a deployment end;
a second absorbent material disposed within the second applicator, the second absorbent material having either a length that is greater than the length of the second sleeve and/or a width that is greater than the diameter of the second sleeve; and
a second plunger for ejecting the second absorbent material from the deployment end of the second applicator;
inserting the deployment end of the second sleeve into the flesh wound;
pressing the second plunger into the plunger end of the second sleeve to eject the second absorbent material out of the deployment end of the second sleeve and into the flesh wound; and
removing the second applicator of the second wound treatment system from the flesh wound, wherein the second absorbent material remains in the flesh wound with the absorbent material from the first wound treatment system.

20. The method of claim 18, further comprising:
packing gauze into the flesh wound after the absorbent material has been ejected into the flesh wound.
